(19) United States
(12) Patent Application Publication
(10) Pub. No.: US 2024/0189248 A1
(43) Pub. Date: Jun. 13, 2024

(54) HIGH EFFICIENCY SURFACE LIGAND EXCHANGE METHODOLOGY FOR HIGH THROUGHPUT PRODUCTION OF PURIFIED DISPERSION COLLOIDAL METAL OXIDES

(71) Applicant: UNIVERSITY OF CENTRAL FLORIDA RESEARCH FOUNDATION, INC., Orlando, FL (US)

(72) Inventors: Sudipta SEAL, Oviedo, FL (US); Craig NEAL, Orlando, FL (US)

(21) Appl. No.: 18/535,535

(22) Filed: Dec. 11, 2023

Related U.S. Application Data
(60) Provisional application No. 63/431,522, filed on Dec. 9, 2022.

(51) Int. Cl. A61K 9/51; A61K 33/00; A61K 33/24; B82Y 5/00; B82Y 40/00

(52) U.S. Cl. CPC A61K 9/5192; A61K 33/00; A61K 33/24; B82Y 5/00; B82Y 40/00

(57) ABSTRACT

The disclosure describes a method of removing and/or replacing an organic ligand from a surface of metal oxide nanoparticles. The method involves mixing a sample of metal oxide nanoparticles comprising an organic ligand in a liquid comprising an organic phase and an aqueous phase, the aqueous phase comprising at least one stripping agent, wherein mixing occurs under conditions to cause the stripping agent to interact with the surface of the metal oxide nanoparticles; removing the aqueous phase from the organic phase following mixing; and optionally, drying the aqueous phase removed from the organic phase.

### BACKGROUND

Colloidal ceria nanoparticles demonstrate substantial promise for applications from catalysis to electronics. In these applications, the key component in nanocrystal performance is their surface energy and related character, as well as effects from quantum confinement

Nanoparticles (NPs) possess a high surface to volume ratio and the surface plays a major role in the interaction between NPs and their environment. Therefore, molecules that bind to their surface are determinative of the utility and efficacy of a given nanoparticle in application.

Organic ligands provide control over nucleation and growth of NPs during synthesis. These ligands are widely used to obtain colloidally stable NPs of controlled size and morphology. Despite allowing significant advantages in NP synthesis, related organic ligands are hydrophobic, obscure surface sites from interaction with the surrounding physicochemical environment, and alter NP crystal surface structure. Accordingly, current processes for particle synthesis in organic solvents with organic ligands produce undesired effects on particle surface chemistry.

### BRIEF DESCRIPTION OF FIGURES

FIG. 1 shows a depiction of general approach for replacement of organic ligands to produce “bare” particle surface.

FIG. 2 shows a stepwise synthesis protocol for making oleyl-CNPs.

FIG. 3 shows protocol for stripping of CNPs oleyl-CNPs and characterization of resulting particles.

FIG. 4 shows x-ray photoelectron spectroscopy graphs of treated nanoparticles.

FIG. 5 shows transmission electron microscopy images of treated nanoparticles.

FIG. 6 sets forth characterization data of nanoparticles treated with a method example described herein.

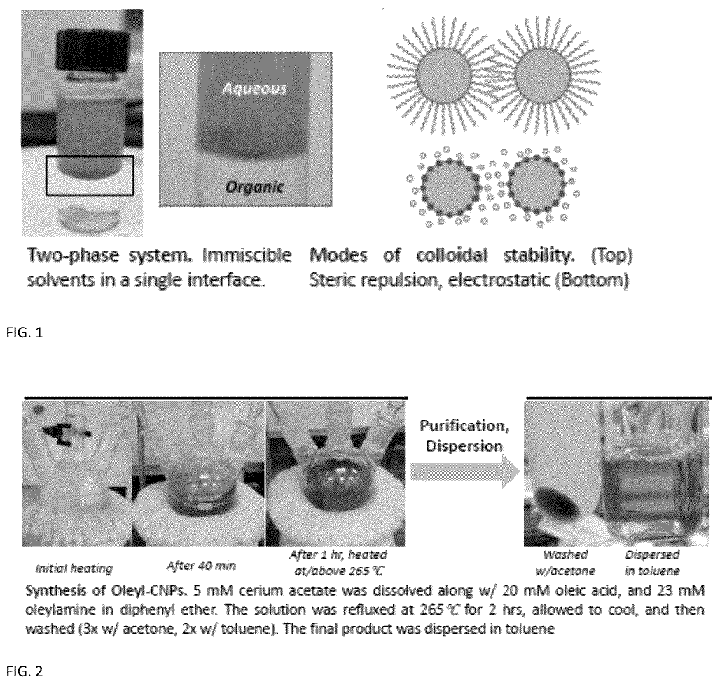

### DETAILED DESCRIPTION

Cerium oxide nanoparticles have the unique property of redox inter conversion between Ce 3+ and Ce 4+ states that is mediated by surface oxygen vacancies according to the following equation:

$$2 {Ce}_{Ce}^{x} + {O}_{o}^{x} {↔}^{{Ce}^{3 +} / {Ce}^{4 +}} {V}_{o}^{••} + 2 {Ce}_{Ce}^{′} + 1 / 2 {O}_{2}$$

The ratio of redox states can be manipulated or varied based on the synthesis method implemented. However, surface reactions between CNPs & small molecules can lead to alterations on the particle surface such as formation or loss of oxygen vacancies.

In certain embodiments, the technology presented herein pertains to a process for the removal of organic ligands, bound to the surface of a metal oxide colloid, and the phase transfer of these particles into an aqueous solution across a two-phase liquid interface. The process is performed by dissolving “stripping agents” (molecules which mediate the removal of surface-bound ligands and may replace these ligands at the particle surface) in an aqueous solution (e.g., de-ionized water, biological buffers) and adding a colloidal suspension immiscible with water over the aqueous solution. From here, the two-phase system is stirred vigorously to allow stripping agent interaction with organic-ligand modified particles in the top phase. Removal of the original ligands, and/or their replacement with ligands miscible in the bottom (aqueous) phase, allows the particles to transfer to the bottom phase.

Process embodiments described herein, facilitate the use of particle synthesis approaches in organic solvents with organic ligands (syntheses which are known to provide control over particle size and shape, with narrow dispersion in these characters) while eliminating several severe drawbacks related to effects on particle surface chemistry. In particular, this process removes organic ligands which show strong toxicity towards biological cells and exposes the material surface to the suspension medium chemical environment, allowing for processes such as catalytic surface reaction.

Metal oxide materials, such as cerium oxide or titanium oxide, may perform valuable surface chemical reactions in biological environment, with the nature of these reactions being sensitive to the materials' surface structure. Further, the ability of such materials to perform these reactions is often ascribable to less stable material features, such as defect structures, which can be irreversibly removed by harsh purification methods (e.g., to remove surface ligands) such as heating or excessive washing. Several stripping agents have been reported in literature for similar methods, such as tetrafluoroborate compounds.

In the case of cerium oxide colloids, the use of ligands with specificity for the cerium oxide surface, such as phosphates and hydrogen peroxide are disclosed. These agents are chosen for their high affinity to the cerium oxide surface as well as their compatibility towards sensitive application environments, such as biological systems. Hydrogen peroxide can be a stripping agent of particular interest for cerium oxide, given its strong affinity for the material surface and the ability of cerium oxide nanomaterials to produce catalytic decomposition of peroxide. Accordingly, in addition to displacement of undesirable organic ligands and phase transfer, peroxide can be degraded in the bottom phase solution to produce bare surfaced particles without altering solution composition post-ligand exchange. In the case of all stripping agents described herein, the aqueous phase is easily isolated by separatory funnel or decanting of the top phase. Further, stripped organic ligands remain dispersed in the top phase: eliminating chances for final sample contamination.

Accordingly, in one embodiment, provided is a method of removing and/or replacing an organic ligand on a surface of metal oxide nanoparticles. The method involves mixing a sample of metal oxide nanoparticles comprising an organic ligand in a liquid comprising an organic phase and an aqueous phase, the aqueous phase comprising at least one stripping agent, wherein mixing occurs under conditions to cause the stripping agent to interact with the surface of the metal oxide nanoparticles. After mixing, the aqueous phase is removed from the organic phase and the aqueous phase is optionally dried. In a specific embodiment, the stripping agent is Na₃PO₄ and/or H₂O₂, wherein the stripping agent also acts as a ligand replacement for the organic ligand. In another specific embodiment, the stripping agent in the liquid is degraded following mixing. The removal of the organic ligand allows the metal oxide particles to transfer from the organic phase to the aqueous phase.

A non-limiting list of stripping agents useful in the method include NaBF₄, Na₃PO₄ and/or H₂O₂. A non-limiting list of metal oxide nanoparticles includes cerium oxide nanoparticles and titanium oxide nanoparticles. Generally, transition metal, lanthanide and actinide oxides may be used as the material for nanoparticles discussed herein.

In select embodiments, the organic phase includes a non-polar solvent or non-polar dispersant. An important property for solvents/dispersants in the organic phase is that they be immiscible with water (i.e., non-polar). A non-limiting list of useful organic solvents includes aromatic solvents such as toluene, xylenes, benzene, mesitylene, and the like and aliphatic solvents such as cyclohexane, hexanes, heptanes, octanes, methylene chloride, methylene bromide, ethylene dichloride, ethylene dibromide, and the like. The aqueous phase may comprise water, and/or any of a number of hydrophilic solvents other than water.

The term “predominant 4+ surface charge” refers to the concentration of cerium ions on the surface and means that the [Ce3+]:[Ce4+] ratio on the surface of the cerium oxide nanoparticle is less than 50%. In a specific example, cerium oxide nanoparticles having a predominant 4+ surface charge have a [Ce3+]:[Ce4+] ratio that is 40% or less.

The term “predominant 3+ surface charge” means that the [Ce3+]:[Ce4+] ratio on the surface of the cerium oxide nanoparticle is greater than 50%. In a specific example, the [Ce3+]:[Ce4+] ratio is greater than 60%.

The term “wet chemical synthesis” refers to a method of making CNPs that involves dissolving a cerium precursor salt in water followed by addition of an oxidizing agent (e.g. hydrogen peroxide). In a specific example, the CNPs are stabilized over a predetermined time period, typically at least 15-30 days.

In one embodiment, disclosed is a method of removing and/or replacing an organic ligand from a surface of metal oxide nanoparticles. The method involves mixing a sample of metal oxide nanoparticles comprising an organic ligand in a liquid comprising an organic phase and an aqueous phase. The aqueous phase includes at least one stripping agent, and mixing occurs under conditions to cause the stripping agent to interact with the surface of the metal oxide nanoparticles. Following mixing, the aqueous phase is removed from the organic phase. In addition, the aqueous phase is dried following removal from the organic phase.

Organic ligands on the surface of metal oxide nanoparticles that are removed or replaced include but are not limited to oleyl, oleylamine, Phospho-, hydroxyl-, or carboxyl-terminated organic species which are (weakly) lipophilic; or Phospho- or carboxyl-functionalized organic species which are (weakly) lipophilic.

The embodiments described herein can be incorporated into any area of industry involving synthesis of nanomaterials. For most of these areas (e.g., medical, optics, electronics) synthesized nanomaterials must be highly reproducible and well-defined. The disclosed embodiments are compatible with the most widely used methods for wet chemical synthesis of such well-defined materials, while allowing for the high-throughput and benign (i.e., not producing irreversible changes to nanomaterial character) purification/isolation. Nanomaterials in technological applications rely on their surface properties; the subject embodiments allow precise control of the material surface by untethering material surface character from the synthesis process.

### Examples

CNPs were modified by removing organic ligands to produce a “bare” particle surface. Chemical stripping agents such as NaBF₄, Na₃PO₄, and H₂O₂; were used based on high affinity towards chemisorption on ceria or their use as stripping agents (ligands w/high affinity for NPs).
Two-phase systems are produced in which there is an oil phase containing CNPs and an aqueous phase containing the stripping agent.
Ligand exchange occurs at the interface as the stripping agent replaces the surface-bound organic ligand, and ultimately, facilitate transfer of CNPs from the oil phase to the aqueous phase

In a specific example, a synthesis method of ultra-small CNP nanodots was implemented, as reported by Krishnan et al. (Krishnan, 2012, incorporated herein by reference), that involves use of diphenyl ether w/oleic acid and oleylamine surface ligands.

Anhydrous Ce(III) acetate was first synthesized from Ce(NO₃)₃·6H₂O as follows:

Generally, cerium nitrate and sodium acetate were dissolved (1:3 molar ratio, respectively) in dH₂O→Ethanol added in 1:1 volume ratio→Cyclohexane added to isolate cerium acetate, and the solution was kept overnight in an oven at 70° C. Aqueous layer was decanted, phase collected, dried, and ground.

FIG. 2 describes a synthesis protocol for synthesizing oleyl-CNPs. 5 mM cerium acetate was dissolved along w/ 20 mM oleic acid, and 23 mM oleylamine in diphenyl ether. The solution was refluxed at 265ºC for 2 hrs, allowed to cool, and then washed (3× w/ acetone, 2× w/ toluene). The final product was dispersed in toluene.

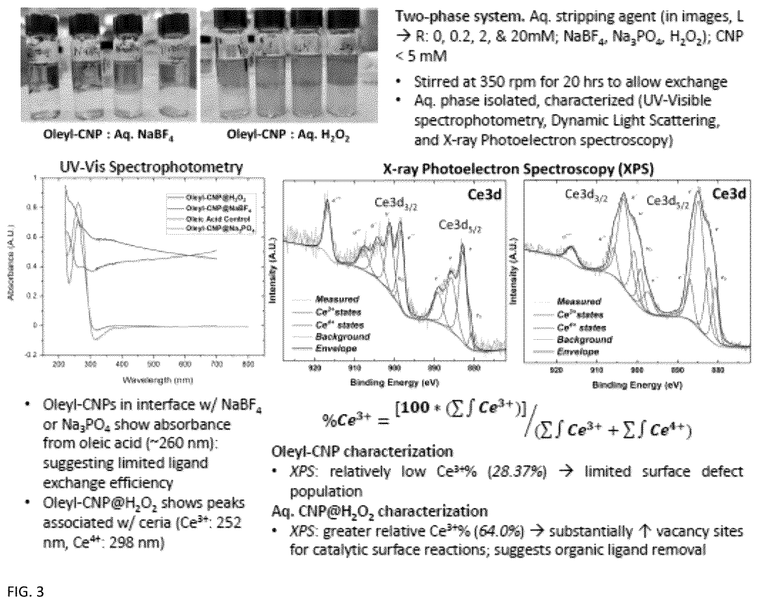

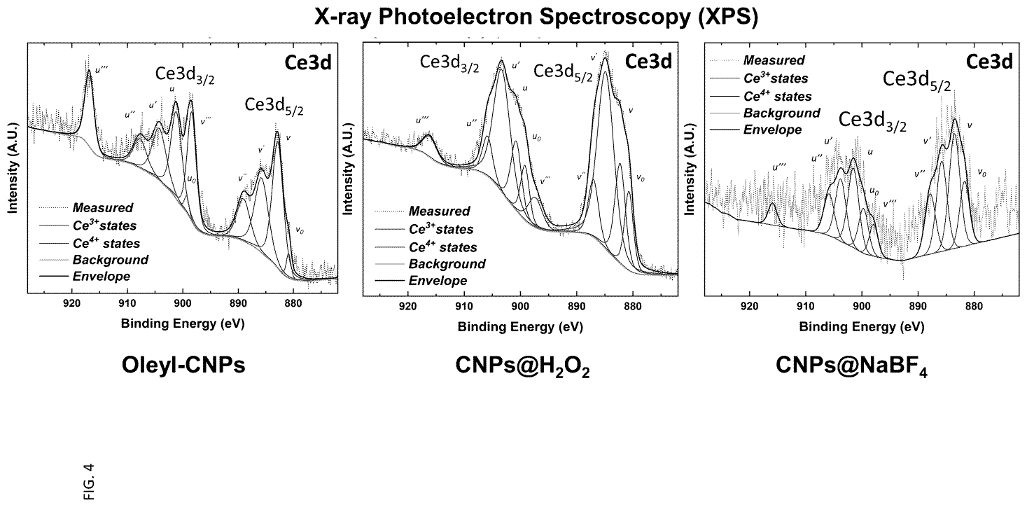

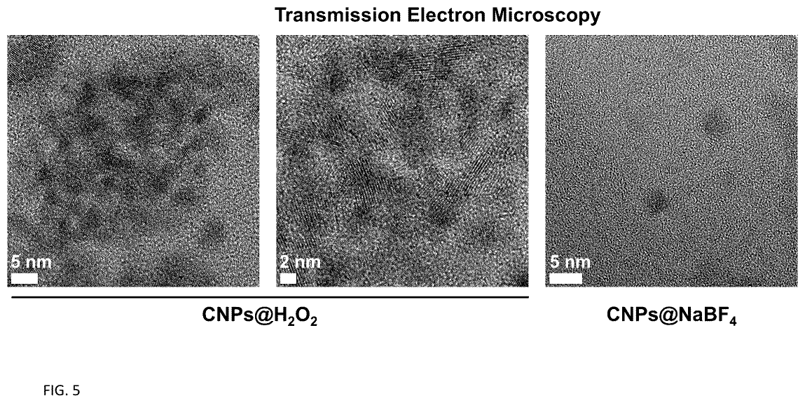

FIG. 3 shows and describes ligand exchange reaction studies. FIG. 4 shows x-ray photoelectron spectroscopy graphs (XPS). (Oleyl-CNPs) Control, as-synthesized cerium oxide nanoparticles. The sample shows a large satellite peak (˜916 nm) which is characteristic of Ce⁴⁺ and is comparatively small in the ligand-stripped samples. (CNPs@H₂O₂, CNPs@NaBF₄) Oleyl-CNPs which have been stripped of oleate ligands by hydrogen peroxide (H₂O₂) or NaBF₄, respectively. Particles from these syntheses show a greater proportion of Ce3+ states relative Ce⁴⁺, as compared with the control formulation. Ce³⁺-sites are localized to the material surface (general property of cerium oxide nanoparticles) and can function as active sites for catalytic reactions (through redox cycling Ce³⁺↔Ce⁴⁺). FIG. 5 shows transmission electron spectroscopy images. (Left, Middle) Oleate-CNPs stripped by H₂O₂ imaged at increasing magnification (L→R), (Right) stripped by NaBF₄. Samples stripped of oleate ligands by either hydrogen peroxide or NaBF₄. Particles show a relatively narrow size dispersion with crystalline character evidenced as lattice fringes (more apparent in images of peroxide-treated particles). Average particle size for particles treated with either stripping agent is ˜3 nm. The small diameter of the particles suggests greater surface activity in potential catalytic reactions due to quantum confinement effects (which become more substantial with narrowing particle spatial dimensions).

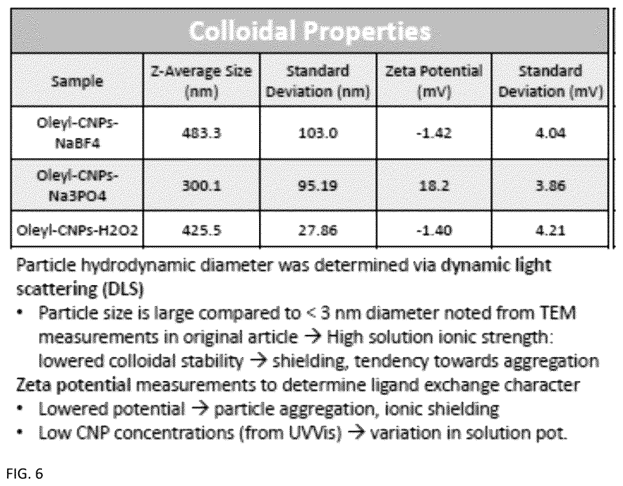

FIG. 6 provides characterization data of particles subjected to stripping protocol outlined in FIG. 3.

### Summary of Results

Particle synthesis was successful as evidenced by solution color changes during synthesis and presence of both 3+ and 4+ redox states in XPS measurements.
Ligand exchange by peroxide appears to show concentration dependence
XPS measurements suggest as-synthesized Oleyl-CNPs have limited reduced sites at the particle surface→likely due to surface capping by organic ligands

XPS measurements of Oleyl-CNP@H₂O₂ show substantial Ce³⁺-content:
Suggests efficacy of ligand stripping, as well as ability of ceria to decompose the peroxy ligand→catalytic degradation of oxidizer (peroxy ligand)
Suggests utility in catalytic applications→e.g., biomedical Nanozyme
Limited efficacy of NaBF₄ and Na₃PO₄ may be ascribed to experimental design→limited interfacial area between aq. and organic phases

### DISCUSSION

Particles treated through our purification process/system embodiments described herein are suitable to create antioxidant particle products for cytoprotective (e.g., protecting healthy cells during radiotherapy treatments for cancer patients) and prooxidant cytotoxic (e.g., inducing cell death in tumor cells) treatments, from the same original particle synthesis products.

### REFERENCES

1. Boles, M. A., Ling, D., Hyeon, T., & Talapin, D. V. (2016). The surface science of nanocrystals. Nature Materials, 15(2), 141-153. https://doi.org/10.1038/nmat4526
2. Asha Krishnan, T. S. S., Eoin Murray, Swapankumar Ghosh. (2012). One-pot synthesis of ultra-small cerium oxide nanodots exhibiting multi-colored fluorescence. Journal of Colloid and Interface Science, 389(1), 16-22. https://doi.org/https://doi.org/10.1016/j.jcis.2012.09.009

Patents providing background on wet chemical synthesis include U.S. Pat. Nos. 8,951,539; 9,415,065, 9,463,437; and 9,161,950 and are incorporated herein by reference in their entirety.

The above description is provided as an aid in examining particular aspects of the invention, and represents only certain embodiments and explanations of embodiments. The examples are in no way meant to be limiting of the invention scope. The materials and methods provided include those which were used in performing the examples above.

It should be borne in mind that all patents, patent applications, patent publications, technical publications, scientific publications, and other references referenced herein are hereby incorporated by reference in this application in order to more fully describe the state of the art to which the present invention pertains.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

What is claimed is:

1. A method of removing and/or replacing an organic ligand from a surface of metal oxide nanoparticles, the method comprising:
mixing a sample of metal oxide nanoparticles comprising an organic ligand in a liquid comprising an organic phase and an aqueous phase, the aqueous phase comprising at least one stripping agent, wherein mixing occurs under conditions to cause the stripping agent to interact with the surface of the metal oxide nanoparticles;
removing the aqueous phase from the organic phase following mixing; and
optionally, drying the aqueous phase removed from the organic phase.

2. The method of claim 1, wherein the stripping agent comprises NaBF₄, Na₃PO₄ and/or H₂O₂.

3. The method of claim 2, wherein the stripping agent comprises Na₃PO₄ and/or H₂O₂ and also acts as a ligand replacement for the organic ligand.

4. The method of any of claim 1, further comprising degrading the stripping agent and/or ligand replacement following mixing.

5. The method of any of claim 1, wherein the metal oxide nanoparticles comprise cerium oxide nanoparticles and/or titanium oxide nanoparticles.

6. The method of any of claim 1, wherein the organic phase comprises a non-polar solvent or dispersant.

7. The method of claim 6, wherein the non-polar solvent or dispersant is selected from aromatic solvents such as toluene, xylenes, benzene, mesitylene, and the like and aliphatic solvents such as cyclohexane, hexanes, heptanes, octanes, methylene chloride, methylene bromide, ethylene dichloride, ethylene dibromide, and the like.

8. The method of any of claim 1, wherein the removal of the organic ligand allows the metal oxide particles to transfer from the organic phase to the aqueous phase.

9. The method of any of claim 1, comprises drying the aqueous phase remove from the organic phase.

10. The method of any of claim 1, wherein the stripping agent is H₂O₂.

11. The method of any of claim 1, wherein the organic ligand comprises a phospho-, hydroxyl-, amino-, or carboxyl-terminated, or functionalized, organic species which is (weakly) lipophilic, optionally, the organic ligand involves oleic acid and/or oleylamine.

12. The method of any of claim 1, wherein the metal oxide nanoparticles in the removed aqueous phase comprises nanoparticles with a predominant 3+ versus 4+ state on their surface.

13. The method of claim 12, wherein the nanoparticles have an average size of approximately 3 to 5 nanometers.

14. The method of claim 1, wherein metal oxide nanoparticles comprise oleyl-CNPs.

15. The method of claim 14, wherein the oleyl-CNPs are synthesized by mixing cerium acetate, oleic acid, and oleylamine in an organic diphenyl ether under conditions to produce oleyl-CNPs; and, optionally, washing the oleyl-CNPs in an organic solvent, and dispersing the oleyl-CNPs in an organic solvent.

16. The method of any of claim 1, wherein the metal oxide nanoparticles comprising an organic ligand comprise a predominant 4+ charge.

17. The method of any of claim 1, wherein the metal oxide nanoparticles in the aqueous phase following the removing step comprise a predominant 3+ charge.
